(19) United States
(12) Patent Application Publication
(10) Pub. No.: US 2025/0308066 A1
(43) Pub. Date: Oct. 2, 2025

(54) POSE ESTIMATION USING INTENSITY THRESHOLDING AND POINT CLOUD ANALYSIS

(71) Applicant: Auris Health, Inc., Santa Clara, CA (US)

(72) Inventors: Mohammad Matinfar, San Jose, CA (US); Jeffrey Adam Kasten, San Mateo, CA (US); Miroslav Drahos, Freehold, NJ (US)

(73) Assignee: Auris Health, Inc., Santa Clara, CA (US)

(21) Appl. No.: 19/088,505

(22) Filed: Mar. 24, 2025

Related U.S. Application Data
(60) Provisional application No. 63/572,032, filed on Mar. 29, 2024.

(51) Int. Cl. G06T 7/73; A61B 1/00; A61B 10/04; G06T 17/00

(52) U.S. Cl. CPC G06T 7/75; A61B 1/00006; A61B 10/04; G06T 17/00; A61B 2010/045; G06T 2207/10028; G06T 2207/10081; G06T 2207/30061; G06T 2210/41; G06T 2210/56

(57) ABSTRACT

This disclosure provides methods, devices, and systems for pose estimation. The present implementations more specifically relate to techniques for determining the pose of a medical instrument within an anatomy. In some aspects, a controller for a medical system may receive image data representing a three-dimensional (3D) model of an anatomy having an instrument disposed therein. The controller generates a point cloud associated with a distal end of the instrument based on the image data and determines a principal axis that maximizes a variance of the point cloud. The controller further determines a pose of the distal end of the instrument based at least in part on the principal axis.

### CROSS-REFERENCE TO RELATED APPLICATIONS

This application claims priority and benefit under 35 U.S.C. § 119 (e) to U.S. Provisional Patent Application No. 63/572,032, filed Mar. 29, 2024, which is incorporated herein by reference in its entirety.

### TECHNICAL FIELD

The present disclosure relates to pose estimation of an object, and specifically to pose estimation using intensity thresholding and point cloud analysis.

### DESCRIPTION OF RELATED ART

During a medical procedure, physicians are presented with various views of a subject's anatomy. The views can include, as few examples, preoperatively generated model views, 3D reconstructed model views, fluoroscopic image views, endoscopic image views, or the like. As part of the medical procedure, physicians may need to accurately identify position and orientation of a depicted object.

### SUMMARY

This Summary is provided to introduce in a simplified form a selection of concepts that are further described below in the Detailed Description. This Summary is not intended to identify key features or essential features of the claimed subject matter, nor is it intended to limit the scope of the claimed subject matter.

One innovative aspect of the subject matter of this disclosure can be implemented in a controller for a medical system, including a processing system and a memory. The memory stores instructions that, when executed by the processing system, cause the controller to receive image data representing a three-dimensional (3D) model of an anatomy having an instrument disposed therein; generate a point cloud associated with a distal end of the instrument based on the image data; determine a first primary axis that maximizes a variance of the point cloud; and determine a pose of the distal end of the instrument based at least in part on the first primary axis.

Another innovative aspect of the subject matter of this disclosure can be implemented in a method of pose estimation. The method includes steps of receiving image data representing a 3D model of an anatomy having an instrument disposed therein; generating a point cloud associated with a distal end of the instrument based on the image data; determining a first primary axis that maximizes a variance of the point cloud; and determining a pose of the distal end of the instrument based at least in part on the first primary axis.

### BRIEF DESCRIPTION OF THE DRAWINGS

Various embodiments are depicted in the accompanying drawings for illustrative purposes and should in no way be interpreted as limiting the scope of the inventions. In addition, various features of different disclosed embodiments can be combined to form additional embodiments, which are part of this disclosure. Throughout the drawings, reference numbers may be reused to indicate correspondence between reference elements.

FIG. 1 illustrates an example medical system, in accordance with one or more examples.

FIG. 2 illustrates a schematic view of different components of the medical system of FIG. 1, in accordance with one or more embodiments.

FIG. 3 illustrates a block diagram depicting various positioning and/or imaging systems/modalities, in accordance with one or more examples.

FIG. 4 illustrates an example block diagram of a tip pose estimation pipeline, in accordance with one or more embodiments.

FIGS. 5A-5C illustrate example images associated with a region extraction process, in accordance with one or more embodiments.

FIG. 6 illustrates an example point cloud, in accordance with one or more embodiments.

FIGS. 7A and 7B illustrate example renderings associated with a scope heading determination process, in accordance with one or more embodiments.

FIG. 8 illustrates an example rendering associated with a scope tip position determination process, in accordance with one or more embodiments.

FIG. 9 illustrates a flow diagram illustrating an instrument pose estimation process, in accordance with one or more embodiments.

FIG. 10 shows a block diagram of an example controller for a medical system, according to some implementations.

FIG. 11 shows an illustrative flowchart depicting an example pose estimation operation, according to some implementations.

### DETAILED DESCRIPTION

The headings provided herein are for convenience only and do not necessarily affect the scope or meaning of the claimed invention. Although certain preferred embodiments and examples are disclosed below, inventive subject matter extends beyond the specifically disclosed embodiments to other alternative embodiments and/or uses and to modifications and equivalents thereof. Thus, the scope of the claims that may arise herefrom is not limited by any of the particular embodiments described below. For example, in any method or process disclosed herein, the acts or operations of the method or process may be performed in any suitable sequence and are not necessarily limited to any particular disclosed sequence. Various operations may be described as multiple discrete operations in turn, in a manner that may be helpful in understanding certain embodiments; however, the order of description should not be construed to imply that these operations are order dependent. Additionally, the structures, systems, and/or devices described herein may be embodied as integrated components or as separate components. For purposes of comparing various embodiments, certain aspects and advantages of these embodiments are described. Not necessarily all such aspects or advantages are achieved by any particular embodiment. Thus, for example, various embodiments may be carried out in a manner that achieves or optimizes one advantage or group of advantages as taught herein without necessarily achieving other aspects or advantages as may also be taught or suggested herein.

Although certain spatially relative terms, such as “outer,” “inner,” “upper,” “lower,” “below,” “above,” “vertical,” “horizontal,” “top,” “bottom,” “lateral,” “upwardly,” “side,” and similar terms, are used herein to describe a spatial relationship of one device/element or anatomical structure to another device/element or anatomical structure, it is understood that these terms are used herein for ease of description to describe the positional relationship between element(s)/structures(s), such as with respect to the illustrated orientations of the drawings. It should be understood that spatially relative terms are intended to encompass different orientations of the element(s)/structures(s), in use or operation, in addition to the orientations depicted in the drawings. For example, an element/structure described as “above” another element/structure may represent a position that is below or beside such other element/structure with respect to alternate orientations of a subject or element/structure, and vice-versa. It should be understood that spatially relative terms, including those listed above, may be understood relative to a respective illustrated orientation of a referenced figure.

Certain reference numbers are re-used across different figures of the figure set of the present disclosure as a matter of convenience for devices, components, systems, features, and/or modules having features that may be similar in one or more respects. However, with respect to any of the embodiments disclosed herein, re-use of common reference numbers in the drawings does not necessarily indicate that such features, devices, components, or modules are identical or similar. Rather, one having ordinary skill in the art may be informed by context with respect to the degree to which usage of common reference numbers can imply similarity between referenced subject matter. Use of a particular reference number in the context of the description of a particular figure can be understood to relate to the identified device, component, aspect, feature, module, or system in that particular figure, and not necessarily to any devices, components, aspects, features, modules, or systems identified by the same reference number in another figure. Furthermore, aspects of separate figures identified with common reference numbers can be interpreted to share characteristics or to be entirely independent of one another. In some contexts, features associated with separate figures that are identified by common reference numbers are not related and/or similar with respect to at least certain aspects.

### Overview

The present disclosure provides systems, devices, and methods for estimating a pose (e.g., position and/or orientation) of a tip of a medical instrument having a flexible elongated body inside of a subject's anatomy from images captured external to the subject. As an example, the tip may be a distal end of an endoscope.

Existing tip pose estimation techniques may not adequately address inaccuracies caused by variations in shape and curvature of the flexible elongated body, geometry of the tip, various imaging artifacts, presence of a medical tool in the working channel of the body, or the like.

The present disclosure discloses an automated or semi-automated process that estimates tip pose in three-dimensional (3D) space. The images may be captured computed tomography (CT) images, including Cone-Beam Computed Tomography (CBCT) images. Such CT images can provide third-person views of the tip pose in contrast to first-person views captured from endoscopic imaging devices inside of the patient. The estimated tip pose can provide another reference point when aligning the tip with a target during a medical procedure. For example, an estimated tip pose based on CT images can aid in aligning a biopsy needle to a nodule during robotically assisted bronchoscopy.

### Example Medical System

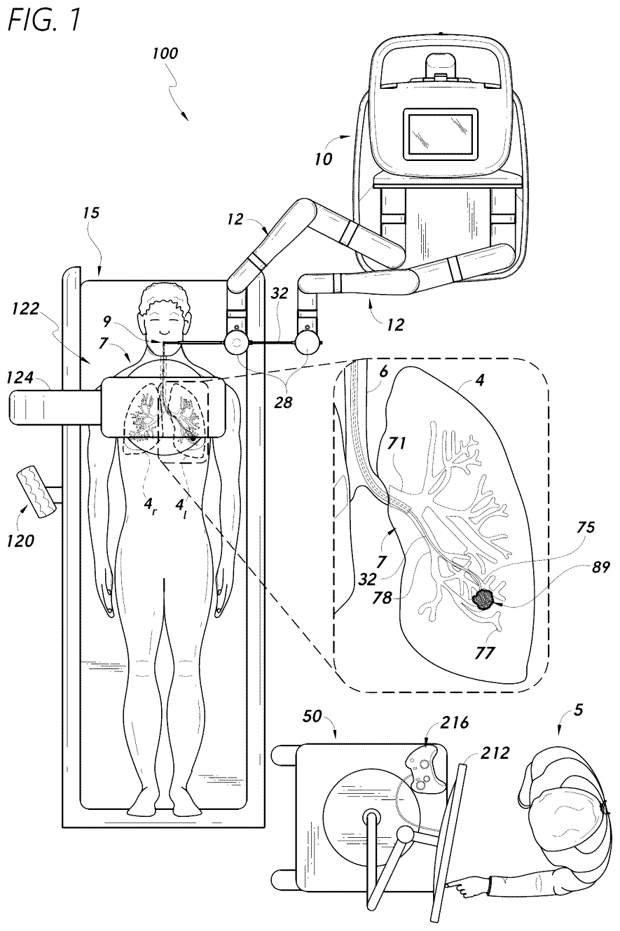

FIG. 1 illustrates an example medical system 100 (also referred to as “surgical medical system 100” or “robotic medical system 100”) in accordance with one or more examples. For example, the medical system 100 can be arranged for diagnostic and/or therapeutic bronchoscopy, as shown. The medical system 100 can include and utilize a robotic system 10, which can be implemented as a robotic cart, for example. Although the medical system 100 is shown as including various cart-based systems/devices, the concepts disclosed herein can be implemented in any type of robotic system/arrangement, such as robotic systems employing rail-based components, table-based robotic end-effectors/manipulators, etc. The robotic system 10 can comprise one or more robotic arms 12 (also referred to as “robotic positioner(s)”) configured to position or otherwise manipulate a medical instrument, such as a medical instrument 32 (e.g., a steerable endoscope or another elongate instrument having a flexible elongated body). For example, the medical instrument 32 can be advanced through a natural orifice access point (e.g., the mouth 9 of a subject 7, positioned on a table 15 in the present example) to deliver diagnostic and/or therapeutic treatment. Although described in the context of a bronchoscopy procedure, the medical system 100 can be implemented for other types of procedures, such as gastro-intestinal (GI) procedures, renal/urological/nephrological procedures, etc. The term “subject” is used herein to refer to live patient as well as any subjects to which the present disclosure may be applicable. For example, the “subject” may refer to subjects including physical anatomic models (e.g., anatomical education model, anatomical model, medical education anatomy model, etc.) used in dry runs, models in computer simulations, or the like that covers non-live patients or test subjects.

With the robotic system 10 properly positioned, the medical instrument 32 can be inserted into the subject 7 robotically, manually, or a combination thereof. In examples, the one or more robotic arms 12 and/or instrument driver(s) 28 thereof can control the medical instrument 32. The instrument driver(s) 28 can be repositionable in space by manipulating the one or more robotic arms 12 into different angles and/or positions.

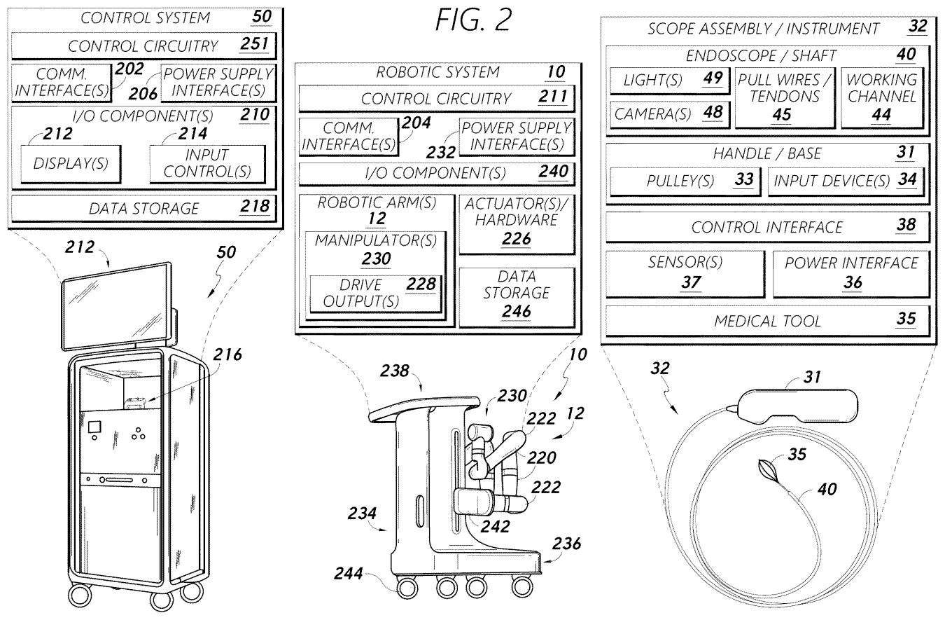

The medical system 100 can also include a control system 50 (also referred to as “control tower” or “mobile tower”), described in detail below with respect to FIG. 2. The control system 50 can include one or more displays 212 to provide/display/present various information related to medical procedures, such as anatomical images. The control system 50 can additionally include one or more control mechanisms, which may be a separate directional input control 216 or a graphical user interface (GUI) presented on the displays 212.

In some embodiments, the display 212 can be a touch-capable display, as shown, that may present anatomical images and allow selection thereon. Few example anatomical images can include CT images, fluoroscopic images, images of an anatomical map, or the like. With the touch-capable display, an operator 5 reviewing the images may find it convenient to identify targets (e.g., target objects or a target region of interest) within the images using a touch-based selection instead of using the directional input control 216. For example, the operator 5 may select a scope tip and/or a nodule using a touchscreen.

The control system 50 can be communicatively coupled (e.g., via wired and/or wireless connection(s)) to the robotic system 10 to provide support for controls, electronics, fluidics, optics, sensors, and/or power to the robotic system 10. Placing such functionality in the control system 50 can allow for a smaller form factor of the robotic system 10 that may be more easily adjusted and/or re-positioned by an operator 5. Additionally, the division of functionality between the robotic system 10 and the control system 50 can reduce operating room clutter and/or facilitate efficient clinical workflow.

The medical system 100 can include an electromagnetic (EM) field generator 120, which is configured to broadcast/emit an EM field that is detected by EM sensors, such as a sensor associated with the medical instrument 32. The EM field can induce small currents in coils of EM sensors (also referred to as “position sensors”), which can be analyzed to determine a pose (position and/or angle/orientation) of the EM sensors relative to the EM field generator 120. In some embodiments, the EM sensors may be positioned at a distal end of the medical instrument 32 and a pose of the distal end may be determined in connection with the pose of the EM sensors. Although EM fields and EM sensors are described in many examples herein, position sensing systems and/or sensors can be any type of position sensing systems and/or sensors, such as optical position sensing systems/sensors, image-based position sensing systems/sensors, etc.

The medical system 100 can further include an imaging system 122 (e.g., a fluoroscopic imaging system) configured to generate and/or provide/send image data (also referred to as “image(s)”) to another device/system. For example, the imaging system 122 can generate image data depicting anatomy of the subject 7 and provide the image data to the control system 50, robotic system 10, a network server, a cloud server, and/or another device. The imaging system 122 can comprise an emitter/energy source (e.g., X-ray source, ultrasound source, or the like) and/or detector (e.g., X-ray detector, ultrasound detector, or the like) integrated into a supporting structure (e.g., mounted on a C-shaped arm support 124), which may provide flexibility in positioning around the subject 7 to capture images from various angles without moving the subject 7. Use of the imaging system 122 can provide visualization of internal structures/anatomy, which can be used for a variety of purposes, such as navigation of the medical instrument 32 (e.g., providing images of internal anatomy to the operator 5), localization of the medical instrument 32 (e.g., based on an analysis of image data), etc. In examples, use of the imaging system 122 can enhance the efficacy and/or safety of a medical procedure, such as a bronchoscopy, by providing clear, continuous visual feedback to the operator 5.

In some examples, the imaging system 122 is a mobile device configured to move around within an environment. For instance, the imaging system 122 can be positioned next to the subject 7 (as illustrated) during a particular phase of a procedure and removed when the imaging system 122 is no longer needed. In other examples, the imaging system 122 can be part of the table 15 or other equipment in an operating environment. The imaging system(s) 122 can be implemented as a Computed Tomography (CT) machine/system, X-ray machine/system, fluoroscopy machine/system, Positron Emission Tomography (PET) machine/system, PET-CT machine/system, CT angiography machine/system, Cone-Beam CT (CBCT) machine/system, 3DRA machine/system, single-photon emission computed tomography (SPECT) machine/system, Magnetic Resonance Imaging (MRI) machine/system, Optical Coherence Tomography (OCT) machine/system, ultrasound machine/system, etc. In some cases, the medical system 100 includes multiple imaging system, such as a first type of imaging system and a second type of imaging system, wherein the different types of imaging systems can be used or positioned over the subject 7 during different phases/portions of a procedure depending on the needs at that time.

In some embodiments, the imaging system 122 can be configured to generate a three-dimensional (3D) model of an anatomy. For example, the imaging system 122 is configured to process multiple images (also referred to as “image data,” in some cases) to generate the 3D model. For example, the imaging device 122 can be implemented as a CT machine configured to capture/generate a series of images/image data (e.g., 2D images/slices) from different angles around the subject 7, and then use one or more algorithms to reconstruct these images/image data into a 3D model. The 3D model can be provided to the control system 50, robotic system 10, a network server, a cloud server, and/or another device, such as for processing, display, or otherwise.

In the interest of facilitating descriptions of the present disclosure, FIG. 1 illustrates a respiratory system as an example anatomy. The respiratory system includes the upper respiratory tract, which comprises the nose/nasal cavity, the pharynx (i.e., throat), and the larynx (i.e., voice box). The respiratory system further includes the lower respiratory tract, which comprises the trachea 6, the lungs 4 (4, and 41), and the various segments of the bronchial tree. The bronchial tree includes primary bronchi 71, which branch off into smaller secondary 78 and tertiary 75 bronchi, and terminate in even smaller tubes called bronchioles 77. Each bronchiole tube is coupled to a cluster of aveoli (not shown). During the inspiration phase of the respiratory cycle, air enters through the mouth and nose and travel down the throat into the trachea 6, into the lungs 4 through the right and left main bronchi 71, into the smaller bronchi airways 78, 75, into the smaller bronchiole tubes 77, and into the alveoli, where oxygen and carbon dioxide exchange takes place.

The bronchial tree is an example luminal network in which robotically-controlled instruments may be navigated and utilized in accordance with the inventive solutions presented here. However, although aspects of the present disclosure are presented in the context of luminal networks including a bronchial network of airways (e.g., lumens, branches) of a subject's lung, some embodiments of the present disclosure can be implemented in other types of luminal networks, such as renal networks, cardiovascular networks (e.g., arteries and veins), gastrointestinal tracts, urinary tracts, etc.

In some embodiments, the imaging system 122 can be configured to capture/update/present images of the anatomy intraoperatively using a CBCT imaging system. During CBCT imaging, the subject 7 may be positioned on the table 15 between an X-ray source and detector mounted on the C-shaped arm support 124 where X-ray beams are passed through a target anatomy, and the resulting images are updated intraoperatively. For example, regarding the lungs 4 of the subject, one or more CBCT captured images or a reconstructed 3D model may be presented to the operator 5 on the display 56. While CBCT is described, it will be understood that the present disclosure contemplates any other imaging techniques capable of providing a 3D reconstruction, such as the normal CT imaging technique.

FIG. 2 illustrates example components of the control system 50, robotic system 10, and medical instrument 32, in accordance with one or more examples. The control system 50 can be coupled to the robotic system 10 and operate in cooperation therewith to perform a medical procedure. For example, the control system 50 can include communication interface(s) 202 for communicating with communication interface(s) 204 of the robotic system 10 via a wireless or wired connection (e.g., to control the robotic system 10). Further, in examples, the control system 50 can communicate with the robotic system 10 to receive position/sensor data therefrom relating to the position of sensors associated with an instrument/member controlled by the robotic system 10. In some examples, the control system 50 can communicate with the EM field generator 120 to control generation of an EM field in an area around a subject 7. The control system 50 can further include a power supply interface(s) 206.

The control system 50 can include control circuitry 251 configured to cause one or more components of the medical system 100 to actuate and/or otherwise control any of the various system components, such as carriages, mounts, arms/positioners, medical instruments, imaging devices, position sensing devices, sensor, etc. Further, the control circuitry 251 can be configured to perform other functions, such as cause display of information, process data, receive input, communicate with other components/devices, and/or any other function/operation discussed herein.

The control system 50 can further include one or more input/out (I/O) components 210 configured to assist a physician or others in performing a medical procedure. For example, the one or more I/O components 210 can be configured to receive input and/or provide output to enable a user to control/navigate the medical instrument 32, the robotic system 10, and/or other instruments/devices associated with the medical system 100. The control system 50 can include one or more displays 212 to provide/display/present various information regarding a procedure. For example, the one or more displays 212 can be used to present navigation information including a virtual anatomical model of anatomy with a virtual representation of a medical instrument, image data, and/or other information. The one or more I/O components 210 can include a user input control(s) 214, which can include any type of user input (and/or output) devices or device interfaces, such as a directional input control(s) 216, touch-based input control(s) including gesture-based input control(s), motion-based input control(s), or the like. The user input control(s) 214 may include one or more buttons, keys, joysticks, handheld controllers (e.g., video-game-type controllers), computer mice, trackpads, trackballs, control pads, sensors (e.g., motion sensors or cameras) that capture hand gestures and finger gestures, touchscreens, toggle (e.g., button) inputs, and/or interfaces/connectors therefore. In examples, such input(s) can be used to generate commands for controlling medical instrument(s), robotic arm(s), and/or other components.

The control system 50 can also include data storage 218 configured to store executable instruments (e.g., computer-executable instructions) that are executable by the control circuitry 251 to cause the control circuitry 251 to perform various operations/functionality discussed herein. In examples, two or more of the components of the control system 50 can be electrically and/or communicatively coupled to each other.

The robotic system 10 can include the one or more robotic arms 12 configured to engage with and/or control, for example, the medical instrument 32 and/or other elements/components to perform one or more aspects of a procedure. As shown, each robotic arm 12 can include multiple segments 220 coupled to joints 222, which can provide multiple degrees of movement/freedom. The robotic system 10 can be configured to receive control signals from the control system 50 to perform certain operations, such as to position one or more of the robotic arms 12 in a particular manner, manipulate an instrument, and so on. In response, the robotic system 10 can control, using control circuitry 211 thereof, actuators 226 and/or other components of the robotic system 10 to perform the operations. For example, the control circuitry 211 can control insertion/retraction, articulation, roll, etc. of a shaft of the medical instrument 32 or another instrument by actuating a drive output(s) 228 of a manipulator(s) 230 (e.g., end-effectors) coupled to a base of a robotically-controllable instrument. The drive output(s) 228 can be coupled to a drive input on an associated instrument, such as an instrument base of an instrument that is coupled to the associated robotic arm 12. The robotic system 10 can include one or more power supply interfaces 232.

The robotic system 10 can include a support column 234, a base 236, and/or a console 238. The console 238 can provide one or more I/O components 240, such as a user interface for receiving user input and/or a display screen (or a dual-purpose device, such as a touchscreen) to provide the physician/user with preoperative and/or intraoperative data. The support column 234 can include an arm support 242 (also referred to as “carriage”) for supporting the deployment of the one or more robotic arms 12. The arm support 242 can be configured to vertically translate along the support column 234. Vertical translation of the arm support 242 allows the robotic system 10 to adjust the reach of the robotic arms 12 to meet a variety of table heights, subject sizes, and/or physician preferences. The base 236 can include wheel-shaped casters 244 (also referred to as “wheels 244”) that allow for the robotic system 10 to move around the operating room prior to a procedure. After reaching the appropriate position, the casters 244 can be immobilized using wheel locks to hold the robotic system 10 in place during the procedure.

The joints 222 of each robotic arm 12 can each be independently-controllable and/or provide an independent degree of freedom available for instrument navigation. In some examples, each robotic arm 12 has seven joints, and thus provides seven degrees of freedom, including “redundant” degrees of freedom. Redundant degrees of freedom can allow robotic arms 12 to be controlled to position their respective manipulators 230 at a specific position, orientation, and/or trajectory in space using different linkage positions and joint angles. This allows for the robotic system 10 to position and/or direct a medical instrument from a desired point in space while allowing the physician to move the joints 222 into a clinically advantageous position away from the patient to create greater access, while avoiding collisions.

The one or more manipulators 230 (e.g., end-effectors) can be couplable to an instrument base/handle, which can be attached using a sterile adapter component in some instances. The combination of the manipulator 230 and coupled instrument base, as well as any intervening mechanics or couplings (e.g., sterile adapter), can be referred to as a manipulator assembly, or simply a manipulator. Manipulator/manipulator assemblies can provide power and/or control interfaces. For example, interfaces can include connectors to transfer pneumatic pressure, electrical power, electrical signals, and/or optical signals from the robotic arm 12 to a coupled instrument base. Manipulator/manipulator assemblies can be configured to manipulate medical instruments (e.g., surgical tools/instruments) using techniques including, for example, direct drives, harmonic drives, geared drives, belts and/or pulleys, magnetic drives, and the like.

The robotic system 10 can also include data storage 246 configured to store executable instruments (e.g., computer-executable instructions) that are executable by the control circuitry 211 to cause the control circuitry 211 to perform various operations/functionality discussed herein. In example, two or more of the components of the robotic system 10 can be electrically and/or communicatively coupled to each other.

Data storage (including the data storage 218, data storage 246, and/or other data storage/memory) can include any suitable or desirable type of computer-readable media. For example, computer-readable media can include one or more volatile data storage devices, non-volatile data storage devices, removable data storage devices, and/or nonremovable data storage devices implemented using any technology, layout, and/or data structure(s)/protocol, including any suitable or desirable computer-readable instructions, data structures, program modules, or other types of data.

Computer-readable media that can include, but is not limited to, phase change memory, static random-access memory (SRAM), dynamic random-access memory (DRAM), other types of random access memory (RAM), read-only memory (ROM), electrically erasable programmable read-only memory (EEPROM), flash memory or other memory technology, compact disk read-only memory (CD-ROM), digital versatile disks (DVD) or other optical storage, magnetic cassettes, magnetic tape, magnetic disk storage or other magnetic storage devices, or any other non-transitory medium that can be used to store information for access by a computing device. As used in certain contexts herein, computer-readable media may not generally include communication media, such as modulated data signals and carrier waves. As such, computer-readable media should generally be understood to refer to non-transitory media.

Control circuitry (including the control circuitry 251, control circuitry 211, and/or other control circuitry) can include circuitry embodied in a robotic system, control system/tower, instrument, or any other component/device. Control circuitry can include any collection of processors, processing circuitry, processing modules/units, chips, dies (e.g., semiconductor dies including one or more active and/or passive devices and/or connectivity circuitry), microprocessors, micro-controllers, digital signal processors, microcomputers, central processing units, field-programmable gate arrays, programmable logic devices, state machines (e.g., hardware state machines), logic circuitry, analog circuitry, digital circuitry, and/or any device that manipulates signals (analog and/or digital) based on hard coding of the circuitry and/or operational instructions. Control circuitry referenced herein can further include one or more circuit substrates (e.g., printed circuit boards), conductive traces and vias, and/or mounting pads, connectors, and/or components. Control circuitry can further comprise one or more storage devices, which may be embodied in a single device, a plurality of devices, and/or embedded circuitry of a device. Such data storage can comprise read-only memory, random access memory, volatile memory, non-volatile memory, static memory, dynamic memory, flash memory, cache memory, data storage registers, and/or any device that stores digital information. In examples in which control circuitry comprises a hardware and/or software state machine, analog circuitry, digital circuitry, and/or logic circuitry, data storage device(s)/register(s) storing any associated operational instructions can be embedded within, or external to, the circuitry comprising the state machine, analog circuitry, digital circuitry, and/or logic circuitry.

Functionality described herein can be implemented by the control circuitry 251 of the control system 50 and/or the control circuitry 211 of the robotic system 10, such as by the control circuitry 251, 211 executing executable instructions to cause the control circuitry 251, 211 to perform the functionality.

The scope assembly/medical instrument 32 includes a handle or base 31 coupled to an endoscope shaft. For example, an endoscope 40 (also referred herein as “scope” or “shaft”) can include the elongate shaft including one or more lights 49 and one or more cameras 48 or other imaging devices. The medical instrument 32 can be powered through a power interface 36 and/or controlled through a control interface 38, each or both of which may interface with a robotic arm/component of the robotic system 10. The medical instrument 32 may further comprise one or more sensors 37, such as pressure sensors and/or other force-reading sensors, which may be configured to generate signals indicating forces experienced at/by one or more components of the medical instrument 32.

The medical instrument 32 includes certain mechanisms for causing the scope 40 to articulate/deflect with respect to an axis thereof. For example, the scope 40 may have been associated with a proximal portion thereof, one or more drive inputs 34 associated, and/or integrated with one or more pulleys/spools 33 that are configured to tension/untension pull wires/tendons 45 of the scope 40 to cause articulation of the scope 40.

The scope 40 can further include one or more working channels 44, which may be formed inside the elongate shaft and run a length of the scope 40. The working channel 44 may serve for deploying therein a medical tool 35 or a component of the medical instrument 32 (e.g., a lithotripter, a basket, forceps, laser, or the like) or for performing irrigation and/or aspiration, out through a distal end of the scope 40, into an operative region surrounding the distal end. The medical instrument 32 may be used in conjunction with a medical tool 35 and include various hardware and control components for the medical tool 35 and, in some instances, include the medical tool 35 as part of the medical instrument 32. For example, as shown, the medical instrument 32 can comprise a basket formed of one or more wire tines but any medical tool 35 are contemplated.

### Mapping, Navigation, and Positioning Modalities/Systems

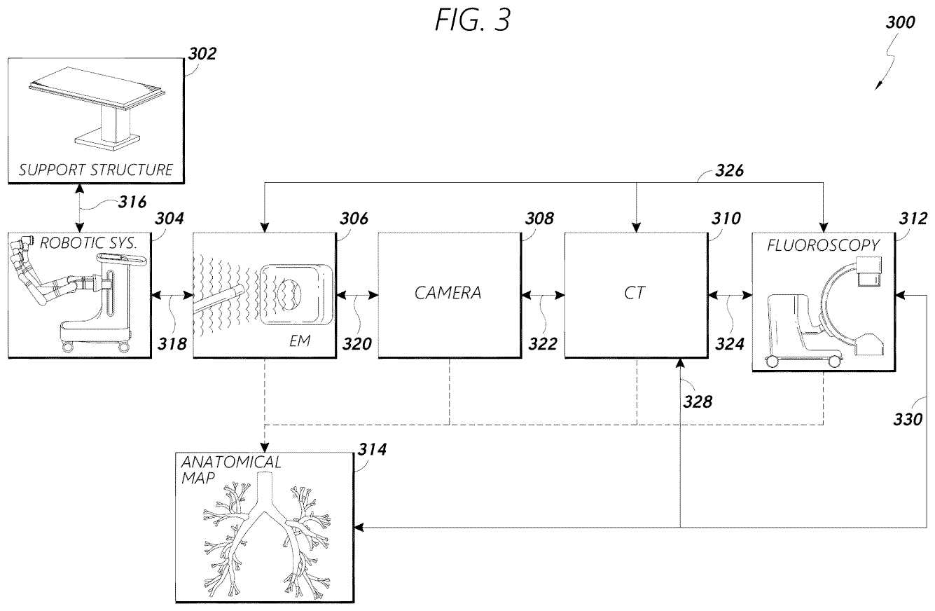

FIG. 3 is a block diagram illustrating a system 300 including various positioning and/or imaging systems/modalities 302-312 (sometimes referred to as “subsystems”), which can be implemented to facilitate anatomical mapping, navigation, positioning, and/or visualization for procedures in accordance with one or more examples. For example, the various systems 302-312 can be configured to provide data for generating an anatomical map, determining a location of an instrument, determining a location of a target, and/or performing other techniques.

Each of the systems 302-312 can be associated with a respective coordinate frame (also referred to as “position coordinate frame”) and/or can provide data/information relating to instrument and/or anatomy locations, wherein registering the various coordinate frames to one another can allow for integration of the various systems to provide mapping, navigation, and/or instrument visualization. For example, registration of various modalities to one another can allow for determined positions in one modality to be tracked and/or superimposed on/in a reference frame associated with another modality, thereby providing layers of positional information that can be combined to provide a robust localization system.

In examples, the system 300 is configured to implement one or more localization/localizing techniques (also referred to as “localization/localizing system 300”). Localization/localizing can refer to processes of determining a location and orientation/pose of an instrument or other element/component within a given space or environment.

In various examples, the anatomical space in which a medical instrument can be localized (i.e., where position and/or shape of the instrument is determined/estimated) is a 2D or 3D portion of a subject's tracheobronchial airways, vasculature, urinary tract, gastrointestinal tract, or any organ or space accessed via lumens. Various modalities can be implemented to provide images/representations/models of the anatomical space using various imaging techniques described in relation to the imaging system 122 of FIG. 1. One or both of preoperative and intraoperative images can be acquired in connection with a procedure.

The systems 302-312 can provide information for generating a 2D or 3D anatomical model/map 314 (e.g., airway model). In examples, the anatomical map 314 and/or other localization information can be displayed to a user, such as the operating user 5, during a procedure to assist the user in perform the procedure. For example, a visualization of a tracked instrument can be superimposed on the anatomical map 314 based on position/sensor data associated with the tracked medical instrument.

As shown, the system 300 can include a surgical bed or other subject platform or positioning/support structure 302 (e.g., the table 15 of FIG. 1). The position of the support structure 302 can be known based on data maintained relating to the position of the support structure 302 within the surgical/procedure environment. Alternatively, or additionally, the position of the support structure 302 can be sensed or otherwise determined using one or more markers and/or an appropriate imaging/positioning modality.

The system 300 can further include a robotic system 304, such as the robotic system 10 (e.g., a robotic cart or other device or system including one or more robotic end effectors). Data relating to the position and/or state of robotic arms, actuators, and/or other components of the robotic system 304 can be known or derived from robotic command data or other robotic data relative to a coordinate frame of the robotic system 304. In some examples, reference frame registration 316 occurs between the support structure 302 and the robotic system 304, which can be a relatively coarse registration (in some cases) based on robotic system/cart-set-up procedure (which can have any suitable or desirable scheme).

The system 300 can further include an electromagnetic (EM) sensor system 306, which can include an EM field generator (e.g., the EM field generator 120) and one or more EM sensors. An EM sensor can be associated with a portion of an instrument that is tracked/controlled, such as along a length of the instrument and/or other elongate member disposed in the working channel of the instrument. In some implementations, the EM field generator can be mechanically coupled to either the support structure 302 or the robotic system 304, in which case registration/association 318 between such systems can be known and/or determined. In some implementations, the registration 318 between the EM sensor system 306 and the robotic system 304 can be determined through forward kinematics and/or field generator mount transform information. For example, the field generator can be mounted to an end effector/manipulator of the robotic system 304, such that the position of the field generator can be known relative to the robotic system positioning frame based on the known relationship between the position of the robotic end effector and the robotic system 304. The EM sensor system 306 can provide instrument pose and/or path information based on sensor readings associated with the instrument.

The system 300 can further include an optical camera system 308 including one or more cameras or other imaging devices, wherein such device(s) is/are configured to generate images of subject anatomy within a visual field thereof, such as real-time image data during a surgical procedure. In examples, registration 320 between the optical camera system 308 and the EM sensor system 306 can be achieved through identification of features having EM sensor data associated therewith, such as by a medical instrument tip, in images generated by the optical camera system 308. The registration 320 can further be based at least in part on hand-eye interaction of the physician when viewing real-time camera images while the EM-sensor-equipped endoscope is navigating in the subject anatomy.

The system 300 can further include a computed tomography (CT) imaging system 310 configured to generate CT images of the subject anatomy, which can be done preoperatively and/or intraoperatively. Specifically, CBCT is a type of CT imaging technique that uses a cone-shaped X-ray beam and a specialized detector to capture multiple two-dimensional (2D) X-ray images from different angles around the patient. In CBCT imaging, a 3D volumetric representation can be reconstructed using the 2D images.

In examples, image processing can be implemented for registration 322 of the CT image data with the camera image data generated by the optical camera system 308. For example, common features identified in both camera image data and CT image data can be identified to relate the CT image frame to the camera image frame in space. In some examples, the CT imaging system 310 can be used to generate preoperative imaging data for producing the anatomical map 314 and/or for path navigation planning.

In examples, the fluoroscopy imaging system 312 can be registered 324 to the CT imaging system 310 using any image processing technique suitable for such registration. Fluoroscopy can provide real-time, continuous X-ray imaging of moving structures inside the body. The images produced during fluoroscopy are typically two-dimensional and show a dynamic view of the area being examined. This real-time imaging can enable the operator 5 to visualize the movement and position of anatomical structures or medical instruments 32 during procedures. Although the CT imaging system 310 and fluoroscopy imaging system 312 are illustrated as separated systems, in examples the same system may perform the functionality of the CT imaging system 310 and fluoroscopy imaging system 312.

In examples, the CT imaging system 310 and/or the fluoroscopy imaging system 312 can be registered 326 to the EM sensor system 306 through various techniques, such as tool registration, a transformation function, etc. In one example, a mechanical structure of the C-arm instrumentation of the system 310, 312 can have a known physical transform/relationship with respect to a mounting position of the EM field generator of the EM sensor system 306. Such known relationship can be used to register the CT/fluoroscopy image space to the EM sensor image space. The connections 328, 330 represent registrations/relationships of the CT imaging system 310 and the fluoroscopy imaging system 312 to the anatomical map 314, respectively.

The position, shape, and/or orientation of an instrument, such as an endoscope, can be determined using any one or more of the systems 302-312, which can facilitate generation of graphical interface data representing the estimated position and/or shape of the instrument relative to the anatomical map 314. The position, shape, and/or orientation of the instrument and/or anatomical map 314 can be displayed on a display device, such as via the control system 50 and/or robotic system 10, or another device. In some examples, the anatomical map 314 also indicates a position(s) of a target(s), such as a location within the anatomy that has been designated for further treatment.

Although the systems 302-312 are discussed in a specific order, the systems can be implemented in different orders. Moreover, the systems can be used in different ways. Further, registration can occur between different systems.

In some illustrations, one or more of the systems 302-312 can be implemented to generate the anatomical map 314 preoperatively and/or determine a location of one or more targets within the anatomical map 314. Intraoperatively, one or more of the systems 302-312 can also be implemented to determine a location of a medical instrument and/or position of a target relative to the anatomical map 314. As discussed herein, one or more of the systems 302-312 can also be implemented to update the anatomical map 314, location of the medical instrument, location of the target, etc.

### Intraoperative Tip Pose Estimation Using External Imaging Modality

Referring back to the respiratory system example of FIG. 1, it was described that the imaging system 122 including an external imaging modality, such as CBCT, may generate a 3D model of the lungs 4. The anatomical map 314 can correspond to a 3D model of a subject's anatomy. Traditionally, the use of external imaging has mostly been limited to the generation of the 3D model for use in preoperative planning and/or as an intraoperative reference model in connection with tip pose estimation of the medical instrument 32.

The EM-based or camera-based tip localization using the anatomical map 314 as a reference model may not be accurate. For example, there may be discrepancies between the anatomical map 314 generated based on preoperatively captured images and actual anatomy of the subject 7 during a medical procedure. As another example, the anatomical map 314 may not capture movement of objects and/or anatomy, such as the respiratory system, gastrointestinal tract, blood vessels, or medical instruments 32. Referring back to FIG. 1, respiration of the subject 7 may cause lung 4 to deform its shape to the extent that the tip may be displaced in the anatomical map 314 at an incorrect location (e.g., a secondary bronchus 78 instead of a tertiary bronchus 75 or in a wrong bronchus).

The present disclosure provides tip pose estimation using images captured during the procedure. CT imaging techniques including CBCT and fluoroscopy can capture or otherwise provide images intraoperatively. These imaging techniques can effectively capture the relationship between the tip of the instrument and its surrounding anatomical environment, providing valuable insight. Additionally, using the disclosed CT images for pose estimation and localization can help resolve discrepancies between the anatomical map 314 and the actual anatomy by providing an additional reference for estimation/localization.

In particular, the present disclosure provides for streamlined CT-based tip pose estimation approaches that are time and resource efficient. The streamlined approaches help address strict latency requirements and heavy resource commitments traditionally involved with CT-based tip pose estimation. Accordingly, the present disclosure contemplates expanding use of the CT-based systems to intraoperative tip pose estimation. The CT-based tip pose estimation can aid the operator 5 in adjusting tip pose when aligning the tip to a target within in 3D space. For example, the CT-based tip pose estimation can help align a scope tip to a nodule (e.g., a nodule 89 in FIG. 1). The CT-based tip pose estimation is described with greater detail below.

### Tip Pose Estimation Pipeline

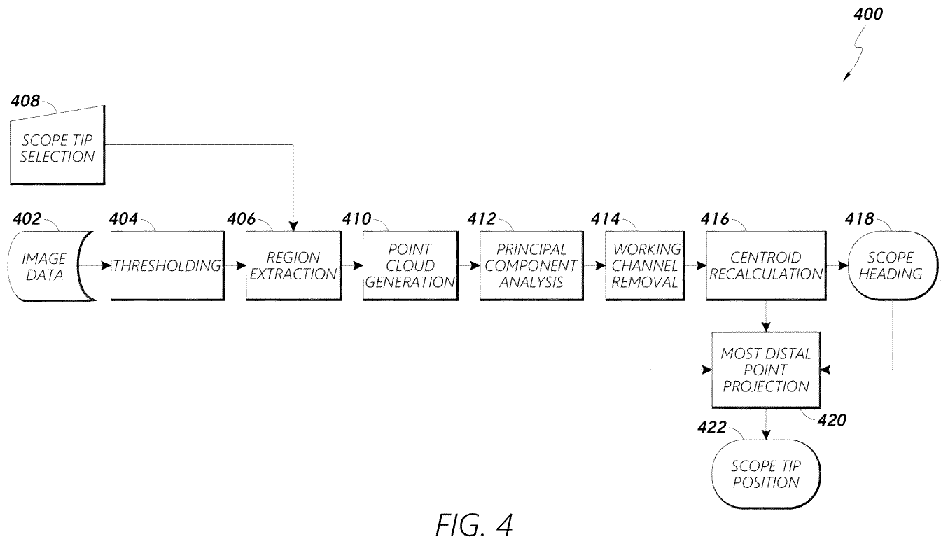

FIG. 4 illustrates an example block diagram of a tip pose estimation pipeline 400, in accordance with one or more embodiments. The pipeline 400 of FIG. 4 visualizes stages or sequences of stages involved in the tip pose estimation process. In particular, the pipeline 400 focuses on data flow and data processing aspects.

As shown, at high level, the pipeline 400 can involve accessing image data 402 as a volumetric representation (e.g., a point cloud or a collection of 2D image slices that collectively provide the volumetric representation), extracting a tip portion from the image data, and analyzing the volumetric representation to determine a tip pose (position and/or orientation). In some implementations, extracting the tip can involve thresholding the image data 402 to differentiate a portion corresponding to a medical instrument from its surrounding anatomy in the image data 402 and extracting the tip from the portion corresponding to the medical instrument. In some implementations, analyzing the volumetric representation can involve performing principal component analysis (“PCA”) and/or calculating a centroid of the point cloud, followed by tip pose determination based on a principal axis and the centroid. It will be understood that, while the present disclosure will be described based on specific implementation techniques mentioned above, other techniques with many variations are possible.

The pipeline 400 can involve accessing (e.g., receiving, retrieving, capturing, decompressing, etc.) image data 402. The image data 402 can correspond to images captured by an external imaging device (e.g., the imaging system 122 positioned external to the subject 7 in FIG. 1) which may be configured for CBCT imaging. In CBCT, a cone-shaped X-ray beam and a specialized detector can capture multiple 2D X-ray images from different angles around a scanned area of the subject 7. A 3D volumetric representation or model of the scanned area may be reconstructed based on the multiple 2D X-ray images. Herein, either the 2D images (that collectively form the 3D representation) or the 3D representation may be referred to as the image data 402 depending on context.

Like in other CT-based images, radiodensity in the image data 402 may be quantified based on Hounsfield unit (HU), which is a measurement scale used to quantify radiodensity of tissues and materials. The Hounsfield unit scale assigns numerical values to different tissues and materials based on their X-ray attenuation properties where metallic objects and dense structures that absorb more X-rays have high HU unit values (more attenuation) in comparison to soft tissues and fluids that absorb less X-rays (lower attenuation). In CT images, denser materials associated with higher HU unit value appears brighter (e.g., with greater brightness intensity) than sparse materials associated with lower HU unit values that appear darker (e.g., with lesser brightness intensity).

At thresholding block 404, the pipeline 400 can involve filtering the image data 402 to differentiate a scope and/or a tip (“scope tip”) thereon from the surrounding anatomy. For example, the filtering of the image data 402 may include comparing the voxels of the image data 402 to at least one threshold value. The threshold value may be a HU unit value or an intensity value (e.g., brightness intensity value) which may be a predefined value or a dynamically computed value based on known (or estimated) properties of the scope. As example HU unit values for a predefined threshold value, a threshold level of +1000 can be associated with gold (Au) or +800 with titanium (Ti). As a dynamically computed value, a HU unit value to be applied to a particular image data 402 may be computed based on global image brightness intensity, which may be helpful to differentiate materials having a widely varying HU unit value range such as ceramics.

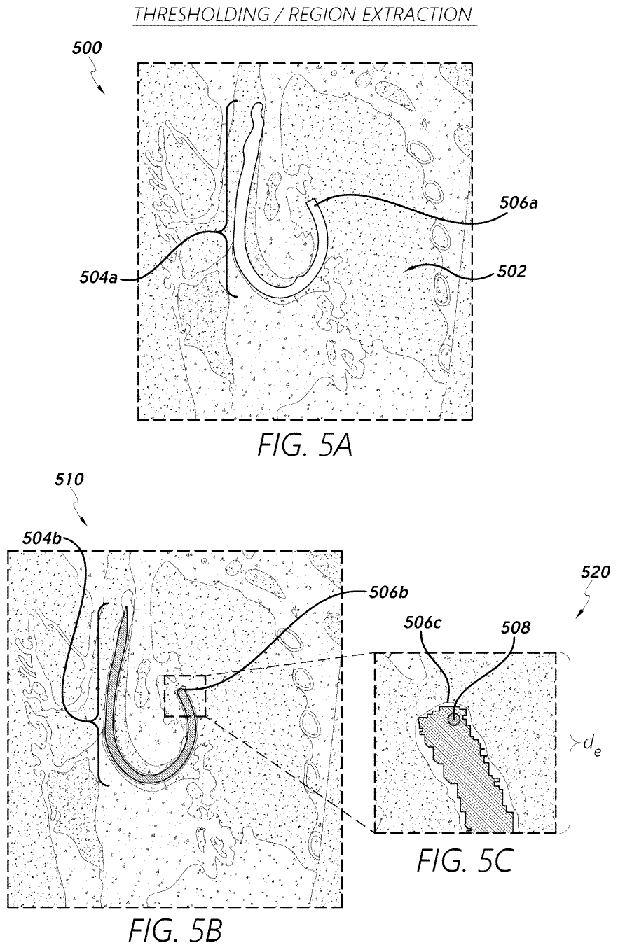

FIGS. 5A-5C illustrate example images associated with a region extraction process, in accordance with one or more embodiments. More specifically, FIGS. 5A-5B illustrate a pre-thresholding image 500 and a post-thresholding image 510, respectively, before and after a thresholding process. The pre-thresholding image 500 depicts surrounding tissue 502, a scope 504a, and a tip 506a thereon. As shown in the post-thresholding image 510, thresholding can differentiate the scope 504b and/or the tip 506b from the surrounding tissue 502 based on a threshold value. In some implementations, the post-thresholding image 510 may be a binary image that represents the scope 504b, the tip 506b, and/or any other metallic objects, with a first value (e.g., “1”) for at-or-above-threshold and a second value (e.g., “0”) for below-threshold. Such thresholding would exclude all tissue 502 in the post-thresholding image 510. The post-thresholding image 510 can be provided to a region extraction block 406.

At region extraction block 406, the pipeline 400 can involve extracting, from a post-thresholding image, a region or volume of interest (also referred herein as “VOI” or “region” for short) including a tip. In the interest of accurately identifying the VOI, in some embodiments, a scope tip selection 408 can be provided to the VOI extraction block 406. For example, an indication (also referred herein as a “seed” or “seed location 508”) corresponding to the tip 506b can be received by the region extraction block 406 for the post-thresholding image 510. The seed location 508 can be an approximate location of the tip 506b provided as user input (such as by the operator 5 of FIG. 1). The manual selection of the seed location 508 may be provided with respect to a presentation of any of the image data 402, the pre-thresholding image 500, and/or the post-thresholding image 510, for example, on a touch-enable display (such as the display 212 of FIG. 2).

A VOI surrounding the seed location may be extracted. FIG. 5C illustrates an example VOI 520 including the tip 506c extracted from the post-thresholding image 510 of FIG. 5B based on the seed location 508. A size of the VOI 520 may be configured based on a geometry of the scope tip. For example, when a scope tip has a known size less than 6 millimeters (mm), the size of the VOI 520 may be predefined as a 10 mm by 10 mm area in 2D space.

The extracted VOI 520 of FIG. 5C is an example showing region extraction from one CT 2D image slice but it will be understood that the region extraction may be performed on a 3D representation (e.g., a 3D reconstructed CBCT image received as the image data 402) in a similar manner. For example, a 3D extracted region, which may be a 10 mm by 10 mm by 10 mm volumetric region surrounding the seed location 508, can be extracted based on the known size of the scope tip and the seed location 508.

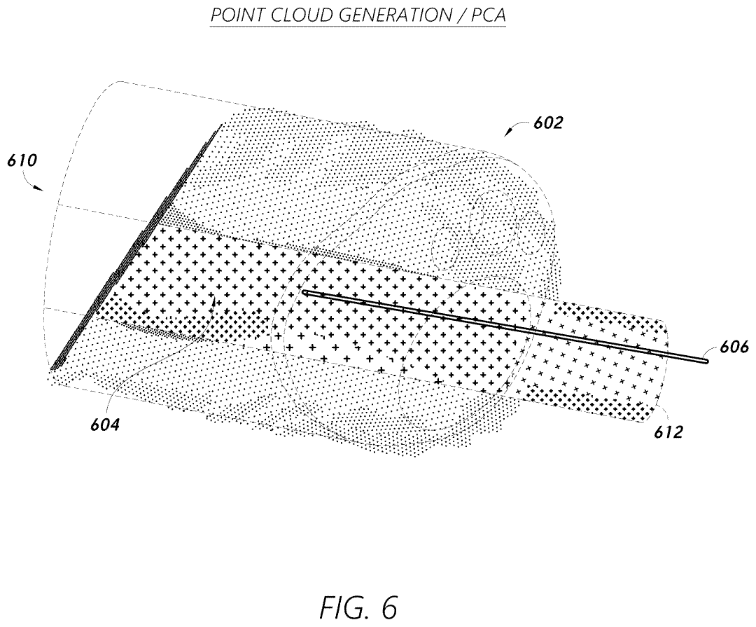

At point cloud generation block 410, the pipeline 400 can involve generation of a point cloud based on an extracted VOI. For example, a point cloud may be generated based on the extracted VOI described in relation to the region extraction block 406. In some examples, points in the point cloud are evenly distributed in 3D and each point may look identical to one another. For instance, each point can correspond to a pixel/voxel in the post-thresholding image 510 that are evenly spaced apart, without differentiating pixels/voxels that correspond to a scope tip from those that correspond to a medical tool. FIG. 6 illustrates an example point cloud 602, in accordance with one or more embodiments. In some implementations, the point cloud 602 can be a collection of all voxels in the extracted VOI that satisfied (or failed to satisfy) the thresholding block 404 of FIG. 4.

In the example of FIG. 6, a silhouette 610 of a 3D model (e.g., a computer-aided design (CAD) model) of the scope tip is overlaid on top of the point cloud 602 to provide a sense of which points of the point cloud 602 correspond to which portion of the scope tip. In particular, the 3D model shows a structure corresponding to working channel 612 (e.g., the working channel 44 of FIG. 2) formed inside and runs along a length of the scope. The point cloud 602 may include medical tool points 604 that map to a medical tool (e.g., the medical tool 35) when the image data 402 from which the point cloud is generated reflects a scope with the medical tool at least partially housed inside the working channel 612. That is, the medical tool points 604 are not part of the scope but are part of the medical tool. To help differentiate the scope tip from the medical tool, each medical tool points 604 is depicted by a ‘+.’ It is noted that the medical tool is made of dense material (e.g., above the threshold value described in relation to the thresholding block 440) which can be extracted as part of the extracted VOI from which medical tool points 604 are generated.

At principal component analysis (PCA) block 412, the pipeline 400 can involve performing a principal component analysis (PCA) to determine one or more principal axes (also known as “principal components”) where a point cloud has the most variance. PCA as applied to a point cloud can find principal axes that capture the directions of highest variance in the point cloud. Of the three principal axes possible for a point cloud in 3D space, a principal axis associated with the maximum variance (also referred to as the “primary axis”) can be selected.

Referring to the point cloud 602, performing PCA can find a primary axis 606 (such as the principal axis which provides the maximum variance in the point cloud). The primary axis 606 is an axis that extends indefinitely and without a directional sense in relation to a reference point. The PCA block 412 may additionally determine a centroid for the point cloud 602, which is akin to a center of mass, and the primary axis 606 can be split into two possible directions, each direction away from the centroid which may be used as the reference point. Either of the two possible directions can correspond to a tip direction (e.g., directional components of a 3D vector). In the present disclosure, the tip direction may be referred as “scope tip heading” or “scope heading,” either of which are reflective of tip orientation.

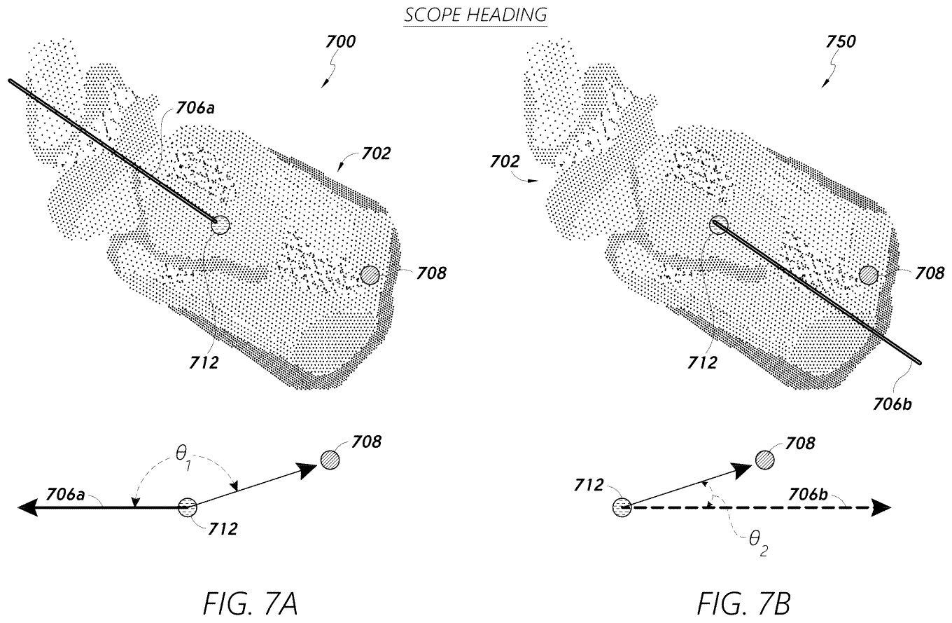

FIGS. 7A and 7B illustrate example renderings 700 and 750, respectively, associated with a scope heading determination process, in accordance with one or more embodiments. More specifically, FIG. 7A shows a point cloud 702 having a centroid 712 with a first possible direction 706a of the scope tip mapped thereto, whereas FIG. 7B shows a second possible direction 706b of the scope tip mapped to the centroid 712 of the point cloud 702. In some implementations, the point cloud 702 may be one example of the point cloud 602 of FIG. 6. To determine the direction in which the scope tip is pointing, the directions 706a and/or 706b can be compared to a reference direction that originates from the centroid 712 and points toward a seed 708 (illustrated by an arrow or vector between the centroid 712 and the seed 708 in FIGS. 7A and 7B). In some implementations, the seed 708 may be one example of the seed location 508 of FIG. 5. For example, the seed 708 may be provided via user input.

The rendering 700 shows the first direction 706a having an offset angle (θ₁) greater than 90 degrees from the reference direction. In contrast, the rendering 750 shows the second direction 706b having an offset angle (θ₂) less than 90 degrees from the reference direction. Accordingly, the second direction 706b is better aligned than first direction 706a with the reference direction. Thus, the second direction 706b may be selected as the direction in which the scope tip is pointing. In a vector-based implementation, the determination of the scope tip direction can be performed based on whether a dot product of the first direction 706a (as a first vector) and the reference direction (as a second vector) is positive or negative. If the dot product is positive, each of the first vector and the second vector has a component in the same direction. In this situation, the first direction 706a can be selected as the scope tip direction. If the dot product is negative, one vector has a component in the opposite direction of the other. In this situation, the direction opposite the first direction 706a (e.g., the second direction 706b) can be selected as the scope tip direction.

The process for determining the scope tip direction can be augmented or refined when a medical tool is disposed inside a working channel of the scope. As described in relation to the point cloud generation block 410 and FIG. 6, a medical tool made of dense material (e.g., materials having radiodensity above the threshold value and surviving the thresholding block 404) and positioned inside a working channel adds to the number of points in the point cloud associated with the scope. These additional points (e.g., the medical tool points 604 represented by “+” in FIG. 6) can extend the point cloud 702 beyond the tip of the scope and, as a result, change the position of the centroid 712. In some implementations, a working channel removal block 414 and centroid re-calculation block 416 may be used to refine the scope tip heading when a medical tool is inserted in the working channel.

At working channel removal block 414, the pipeline 400 can involve filtering or removing medical tool points from a point cloud. Referring back to the example of FIG. 6, the medical tool points 604 (e.g., the “+” points) can be removed from the point cloud 602. In some implementations, the removal of the medical tool points 604 can be based on a known geometry of the working channel 612. For example, when the working channel 612 has a known diameter, assuming that principal axes are aligned with a center line of the scope tip, the working channel removal block 414 can remove each of the medical tool points 604 from the point cloud 602. After removing the medical tool points 604, the point cloud 602 may become a hollow cylindrical point cloud (e.g., leaving a hollow cylinder where the medical tool points 604 once were).

At centroid recalculation block 416, the pipeline 400 can involve recalculating a revised centroid for the point cloud after filtering or removing the medical tool points in the working channel. For example, the centroid can be recalculated for the hollow cylindrical point cloud after the working channel removal block 414. The recalculated centroid more accurately reflects the center of mass of the scope tip compared to the centroid calculated at the PCA block 412.

In some embodiments, the scope heading may be updated as an updated scope heading 418 in FIG. 4 based on the recalculated centroid. In some embodiments, the updated scope heading 418 can be represented as a vector originating from the recalculated centroid, aligning with a primary axis of the filtered point cloud (such as the principal axis having the maximum variance), and pointing toward a selected scope tip direction on the primary axis (such as described with reference to FIGS. 7A and 7B). Thus, the recalculated centroid can help refine the scope tip position to provide an updated scope tip position 422.

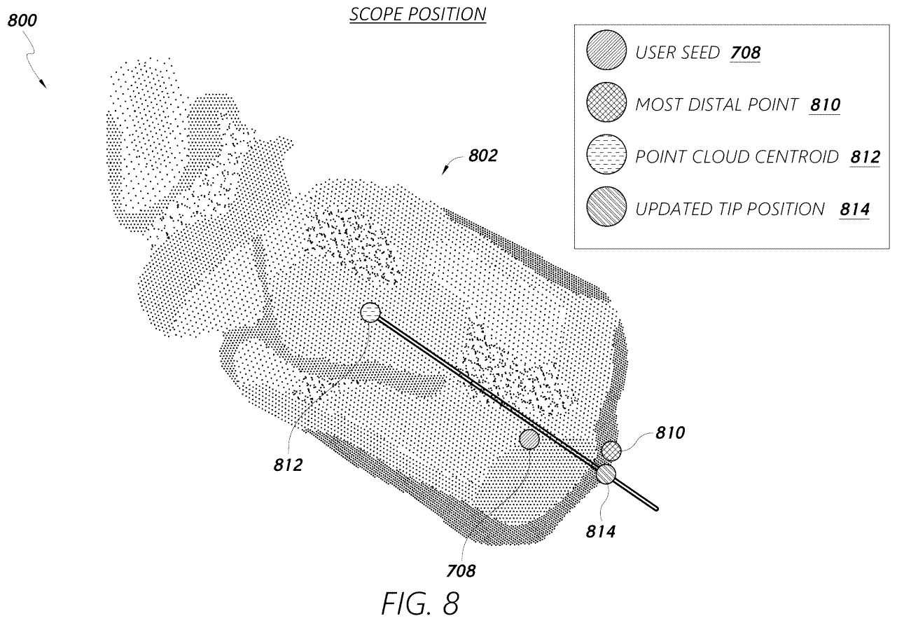

At most distal point projection block 420, the pipeline 400 can involve projecting the most distal point in the point cloud onto its primary axis. For example, FIG. 8 illustrates an example rendering 800 associated with a scope tip position determination process, in accordance with one or more embodiments. More specifically, FIG. 8 shows a point cloud 802 having a centroid 812 and a most distal point 810 projected onto its primary axis. In some embodiments, the most distal point 810 in the point cloud 802 can be selected as a farthest point from the centroid 812 in the point cloud 802 that is near the seed location 708 (such as within a threshold proximity of the seed location 708). The projection of the most distal point 810 onto the primary axis can be used to determine an updated tip position 814 on the primary axis as the updated scope tip position 422.

The scope heading can represent tip orientation, and the scope tip position can represent the tip position. Accordingly, the pipeline 400 can estimate the scope tip pose using externally captured images. It will be understood that depicted blocks in the pipeline 400 are merely exemplary, and that fewer or more blocks, as well as blocks organized in different orders, may be involved in the pose estimation for the scope tip. For instance, where there is no medical tool involved, the working channel removal block 414 and the centroid recalculation block 416 may be skipped. Many other variations are also possible.

### Instrument Pose Estimation Workflow

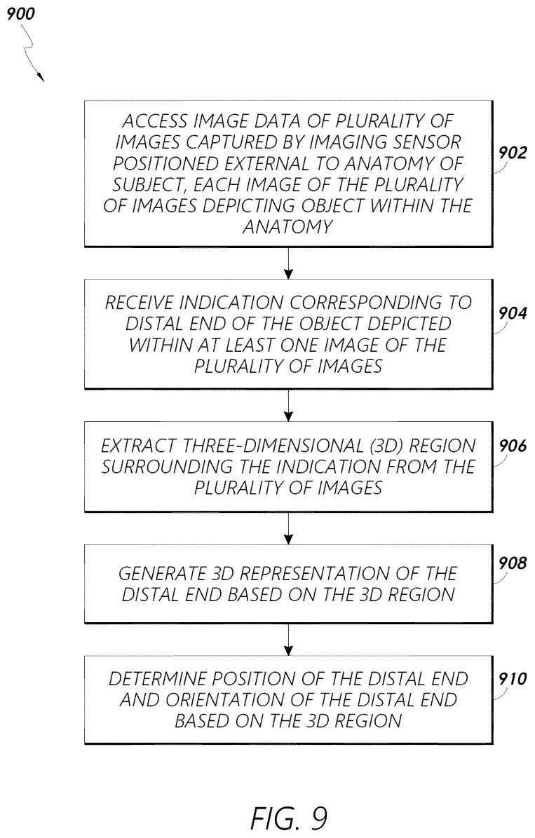

FIG. 9 illustrates a flow diagram illustrating an instrument pose estimation process 900, in accordance with one or more embodiments. In some implementations, the instrument may be a scope (such as the scope 40 of FIGS. 1 and 2). The process 900, when followed, can determine scope heading and scope tip position from externally captured images.

At block 902, the process 900 can involve accessing image data of a plurality of images captured by an imaging sensor positioned external to an anatomy of a subject. The images can be CT images including CBCT images. Each image of the plurality of images can depict an object within the anatomy.

At block 904, the process 900 can involve receiving an indication corresponding to a distal end of the object depicted within at least one image of the plurality of images. The object can be a scope and the distal end can be a tip of the scope. In some embodiments, an operator may manually provide the indication by selecting one or more pixels of the presented image. The selection can be made through various input controls including a touch-enabled display, as shown in FIG. 1.

At block 906, the process 900 can involve extracting a VOI surrounding the indication from the images. The VOI may be extracted based on a known geometry of the distal end of the object. For example, a scope tip may have a dimension less than 5.7 millimeters in diameter and the extracted VOI may be configured as 10 mm by 10 mm by 10 mm.

At block 908, the process 900 can involve generating a 3D representation of the distal end based on the VOI. The 3D representation can be a point cloud of all points known to be associated with the object or the distal end thereof. Whether a point is associated with the object or not may be determined based on various imaging processing techniques including thresholding.

At block 910, the process 900 can involve determining a position of the distal end and an orientation of the distal end based on the VOI. The orientation may be determined as a vector originating at a reference point and having a direction pointing outward from the distal end. The reference point may be a centroid of the 3D representation. The direction may be determined using PCA on the 3D representation and filtered/selected based on the indication. The position may be determined by projecting the most distal point (farthest from the centroid) onto the primary axis determined by the PCA.

It will be understood that depicted blocks in the process 900 are exemplary and fewer or more blocks, as well as blocks organized in different orders, may be involved in the tip pose estimation. Many variations are possible.

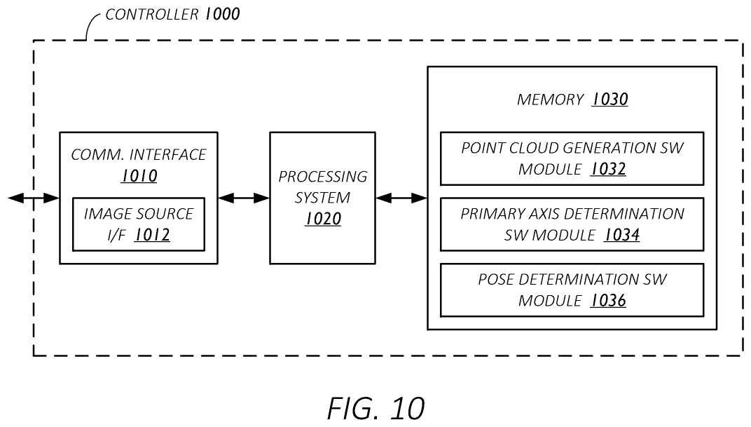

FIG. 10 shows another block diagram of an example controller 1000 for a medical system, according to some implementations. In some implementations, the controller 1000 may be one example of the control circuitry 251 and/or 211 of FIG. 2. More specifically, the controller 1000 is configured to estimate the pose of an object within an anatomy. With reference for example to FIG. 4, the controller 100 may implement one or more stages of the tip pose estimation pipeline 400.

The controller 1000 includes a communication interface 1010, a processing system 1020, and a memory 1030. The communication interface 1010 is configured to communicate with one or more components of the medical system. More specifically, the communication interface 1010 includes an image source interface (I/F) 1012 for communicating with one or more image sources (such as the CT imaging system 310 and/or the fluoroscopy imaging system 312 of FIG. 3). In some implementations, the image source I/F 1012 may receive image data representing a 3D model of an anatomy having an instrument disposed therein.

The memory 1030 may include a non-transitory computer-readable medium (including one or more nonvolatile memory elements, such as EPROM, EEPROM, Flash memory, or a hard drive, among other examples) that may store the following software (SW) modules: a point cloud generation SW module 1032 to generate a point cloud associated with a distal end of the instrument based on the image data; a primary axis determination SW module 1034 to determine a primary axis that maximizes a variance of the point cloud; and a pose determination SW module 1036 to determine a pose of the distal end of the instrument based at least in part on the primary axis.

The processing system 1020 may include any suitable one or more processors capable of executing scripts or instructions of one or more software programs stored in the controller 1000 (such as in the memory 1030). For example, the processing system 1020 may execute the point cloud generation SW module 1032 to generate a point cloud associated with a distal end of the instrument based on the image data. The processing system 1020 also may execute the primary axis determination SW module 1034 to determine a primary axis that maximizes a variance of the point cloud. The processing system 1020 may further execute the pose determination SW module 1036 to determine a pose of the distal end of the instrument based at least in part on the primary axis.

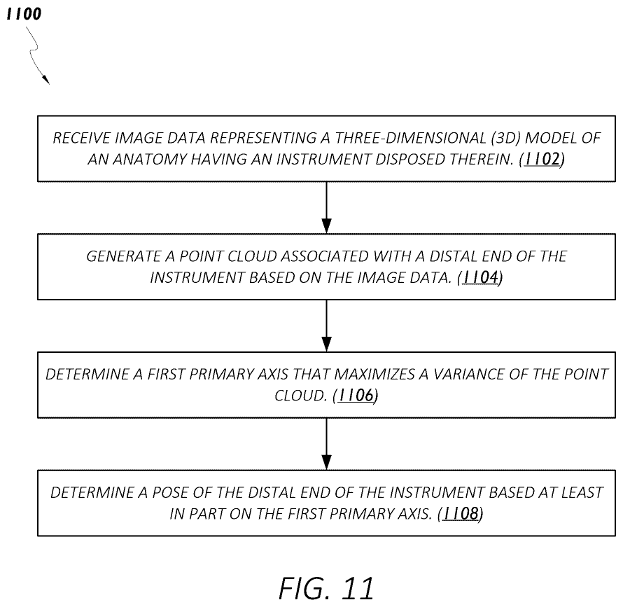

FIG. 11 shows an illustrative flowchart depicting an example pose estimation operation 1100, according to some implementations. In some implementations, the example operation 1100 may be performed by a controller for a medical system such as the controller 1000 of FIG. 10.

The controller receives image data representing a 3D model of an anatomy having an instrument disposed therein (1302). The controller generates a point cloud associated with a distal end of the instrument based on the image data (1304). The controller determines a first primary axis that maximizes a variance of the point cloud (1306). The controller further determines a pose of the distal end of the instrument based at least in part on the first primary axis (1308).

In some aspects, the controller may further filter the image data based on one or more known properties of the instrument so that the filtered image data includes voxels associated with the instrument and excludes voxels associated with the anatomy, where the point cloud is generated based on the filtered image data. In some implementations, the filtering of the image data may include comparing an intensity of each voxel of the image data to a threshold intensity associated with the one or more known properties of the instrument and assigning a binary value to each voxel of the image data based on whether the intensity of the voxel exceeds the threshold intensity.

In some aspects, the controller may further receive a seed location associated with the distal end of the instrument in the 3D model and extract a volume of interest (VOI) surrounding the seed location in the 3D model, where the point cloud is generated based on the VOI. In some implementations, the VOI may be configured based on a known geometry of the instrument.

In some aspects, the controller may further identify a portion of the point cloud associated with a working channel of the instrument, remove the identified portion from the point cloud, and determine a second primary axis that maximizes a variance of the point cloud having the portion associated with the working channel removed, where the pose of the distal end of the instrument is determined based at least in part on the second primary axis. In some implementations, the portion of the point cloud associated with the working channel may be identified based on the first primary axis, a centroid of the point cloud, and a known diameter of the working channel.

In some aspects, the determining of the pose of the distal end of the instrument may include determining a reference direction associated with an orientation of the distal end of the instrument, comparing the reference direction to a first direction along the first primary axis originating from a centroid of the point cloud, comparing the reference direction to a second direction along the first primary axis opposite the first direction, and determining the orientation of the distal end of the instrument based on comparing the reference direction to the first direction and the second direction. In some implementations, the determining of the reference direction may include receiving a seed location associated with the distal end of the instrument in the 3D model and determining a vector pointing from the centroid of the point cloud to the seed location, the vector representing the reference direction.

In some aspects, the determining of the pose of the distal end of the instrument may include receiving a seed location associated with the distal end of the instrument in the 3D model, selecting a point in the point cloud furthest from a centroid of the point cloud based on the seed location, and projecting the selected point onto the first primary axis, where the projected point represents a position of the distal end of the instrument.

### Additional Embodiments

Depending on the embodiment, certain acts, events, or functions of any of the processes or algorithms described herein can be performed in a different sequence, may be added, merged, or left out altogether. Thus, in certain embodiments, not all described acts or events are necessary for the practice of the processes.

Conditional language used herein, such as, among others, “can,” “could,” “might,” “may,” “e.g.,” and the like, unless specifically stated otherwise, or otherwise understood within the context as used, is intended in its ordinary sense and is generally intended to convey that certain embodiments include, while other embodiments do not include, certain features, elements and/or steps. Thus, such conditional language is not generally intended to imply that features, elements and/or steps are in any way required for one or more embodiments or that one or more embodiments necessarily include logic for deciding, with or without author input or prompting, whether these features, elements and/or steps are included or are to be performed in any particular embodiment. The terms “comprising,” “including,” “having,” and the like are synonymous, are used in their ordinary sense, and are used inclusively, in an open-ended fashion, and do not exclude additional elements, features, acts, operations, and so forth. Also, the term “or” is used in its inclusive sense (and not in its exclusive sense) so that when used, for example, to connect a list of elements, the term “or” means one, some, or all of the elements in the list. Conjunctive language such as the phrase “at least one of X, Y and Z,” unless specifically stated otherwise, is understood with the context as used in general to convey that an item, term, element, etc. may be either X, Y or Z. Thus, such conjunctive language is not generally intended to imply that certain embodiments require at least one of X, at least one of Y and at least one of Z to each be present.

It should be appreciated that in the above description of embodiments, various features are sometimes grouped together in a single embodiment, figure, or description thereof for the purpose of streamlining the disclosure and aiding in the understanding of one or more of the various inventive aspects. This method of disclosure, however, is not to be interpreted as reflecting an intention that any claim require more features than are expressly recited in that claim. Moreover, any components, features, or steps illustrated and/or described in a particular embodiment herein can be applied to or used with any other embodiment(s). Further, no component, feature, step, or group of components, features, or steps are necessary or indispensable for each embodiment. Thus, it is intended that the scope of the inventions herein disclosed and claimed below should not be limited by the particular embodiments described above, but should be determined only by a fair reading of the claims that follow.

It should be understood that certain ordinal terms (e.g., “first” or “second”) may be provided for ease of reference and do not necessarily imply physical characteristics or ordering. Therefore, as used herein, an ordinal term (e.g., “first,” “second,” “third,” etc.) used to modify an element, such as a structure, a component, an operation, etc., does not necessarily indicate priority or order of the element with respect to any other element, but rather may generally distinguish the element from another element having a similar or identical name (but for use of the ordinal term). In addition, as used herein, indefinite articles (“a” and “an”) may indicate “one or more” rather than “one.” Further, an operation performed “based on” a condition or event may also be performed based on one or more other conditions or events not explicitly recited.

Unless otherwise defined, all terms (including technical and scientific terms) used herein have the same meaning as commonly understood by one of ordinary skill in the art to which example embodiments belong. It is further understood that terms, such as those defined in commonly used dictionaries, should be interpreted as having a meaning that is consistent with their meaning in the context of the relevant art and not be interpreted in an idealized or overly formal sense unless expressly so defined herein. As used herein, the term “patient” may generally refer to humans, anatomical models, simulators, cadavers, and other living or non-living objects.

The spatially relative terms “outer,” “inner,” “upper,” “lower,” “below,” “above,” “vertical,” “horizontal,” and similar terms, may be used herein for ease of description to describe the relations between one element or component and another element or component as illustrated in the drawings. It be understood that the spatially relative terms are intended to encompass different orientations of the device in use or operation, in addition to the orientation depicted in the drawings. For example, in the case where a device shown in the drawing is turned over, the device positioned “below” or “beneath” another device may be placed “above” another device. Accordingly, the illustrative term “below” may include both the lower and upper positions. The device may also be oriented in the other direction, and thus the spatially relative terms may be interpreted differently depending on the orientations.

Unless otherwise expressly stated, comparative and/or quantitative terms, such as “less,” “more,” “greater,” and the like, are intended to encompass the concepts of equality. For example, “less” can mean not only “less” in the strictest mathematical sense, but also, “less than or equal to.”

What is claimed is:

1. A controller for a medical system, comprising:
a processing system; and
a memory storing instructions that, when executed by the processing system, cause the controller to:
receive image data representing a three-dimensional (3D) model of an anatomy having an instrument disposed therein;
generate a point cloud associated with a distal end of the instrument based on the image data;
determine a first primary axis that maximizes a variance of the point cloud; and
determine a pose of the distal end of the instrument based at least in part on the first primary axis.

2. The controller of claim 1, wherein execution of the instructions further causes the controller to:
filter the image data based on one or more known properties of the instrument so that the filtered image data includes voxels associated with the instrument and excludes voxels associated with the anatomy, the point cloud generated based on the filtered image data.

3. The controller of claim 2, wherein the filtering of the image data comprises:
comparing an intensity of each voxel of the image data to a threshold intensity associated with the one or more known properties of the instrument; and
assigning a binary value to each voxel of the image data based on whether the intensity of the voxel exceeds the threshold intensity.

4. The controller of claim 1, wherein execution of the instructions further causes the controller to:
receive a seed location associated with the distal end of the instrument in the 3D model; and
extract a volume of interest (VOI) surrounding the seed location in the 3D model, the point cloud generated based on the VOI.

5. The controller of claim 4, wherein the VOI is configured based on a known geometry of the instrument.

6. The controller of claim 1, wherein execution of the instructions further causes the controller to:
identify a portion of the point cloud associated with a working channel of the instrument;
remove, from the point cloud, the portion associated with the working channel; and
determine a second primary axis that maximizes a variance of the point cloud having the portion associated with the working channel removed, the pose of the distal end of the instrument determined based at least in part on the second primary axis.

7. The controller of claim 6, wherein the portion of the point cloud associated with the working channel is identified based on the first primary axis, a centroid of the point cloud, and a known diameter of the working channel.

8. The controller of claim 1, wherein the determining of the pose of the distal end of the instrument comprises:
determining a reference direction associated with an orientation of the distal end of the instrument;
comparing the reference direction to a first direction along the first primary axis originating from a centroid of the point cloud;
comparing the reference direction to a second direction along the first primary axis originating from the centroid of the point cloud; and
determining the orientation of the distal end of the instrument based on comparing the reference direction to the first direction and the second direction.

9. The controller of claim 8, wherein the determining of the reference direction comprises:
receiving a seed location associated with the distal end of the instrument in the 3D model; and
determining a vector pointing from the centroid of the point cloud to the seed location, the vector representing the reference direction.

10. The controller of claim 1, wherein the determining the pose of the distal end of the instrument comprises:
receiving a seed location associated with the distal end of the instrument in the 3D model;
selecting a point in the point cloud furthest from a centroid of the point cloud based on the seed location; and
projecting the selected point onto the first primary axis, the projected point representing a position of the distal end of the instrument.

11. A method of pose estimation, comprising:
receiving image data representing a three-dimensional (3D) model of an anatomy having an instrument disposed therein;
generating a point cloud associated with a distal end of the instrument based on the image data;
determining a first primary axis that maximizes a variance of the point cloud; and
determining a pose of the distal end of the instrument based at least in part on the first primary axis.

12. The method of claim 11, further comprising:
filtering the image data based on one or more known properties of the instrument so that the filtered image data includes voxels associated with the instrument and excludes voxels associated with the anatomy, the point cloud generated based on the filtered image data.

13. The method of claim 12, wherein the filtering of the image data comprises:
comparing an intensity of each voxel of the image data to a threshold intensity associated with the one or more known properties of the instrument; and
assigning a binary value to each voxel of the image data based on whether the intensity of the voxel exceeds the threshold intensity.

14. The method of claim 11, further comprising:
receiving a seed location associated with the distal end of the instrument in the 3D model; and
extracting a volume of interest (VOI) surrounding the seed location in the 3D model, the point cloud generated based on the VOI.

15. The method of claim 14, wherein the VOI is configured based on a known geometry of the instrument.

16. The method of claim 11, further comprising:
identifying a portion of the point cloud associated with a working channel of the instrument;
removing, from the point cloud, the portion associated with the working channel; and
determining a second primary axis that maximizes a variance of the point cloud having the portion associated with the working channel removed, the pose of the distal end of the instrument determined based at least in part on the second primary axis.

17. The method of claim 16, wherein the portion of the point cloud associated with the working channel is identified based on the first primary axis, a centroid of the point cloud, and a known diameter of the working channel.

18. The method of claim 11, wherein the determining of the pose of the distal end of the instrument comprises:
determining a reference direction associated with an orientation of the distal end of the instrument;
comparing the reference direction to a first direction along the first primary axis originating from a centroid of the point cloud;
comparing the reference direction to a second direction along the first primary axis opposite the first direction; and
determining the orientation of the distal end of the instrument based on comparing the reference direction to the first direction and the second direction.

19. The method of claim 18, wherein the determining of the reference direction comprises:
receiving a seed location associated with the distal end of the instrument in the 3D model; and
determining a vector pointing from the centroid of the point cloud to the seed location, the vector representing the reference direction.

20. The method of claim 11, wherein the determining of the pose of the distal end of the instrument comprises:
receiving a seed location associated with the distal end of the instrument in the 3D model;
selecting a point in the point cloud furthest from a centroid of the point cloud based on the seed location; and
projecting the selected point onto the first primary axis, the projected point representing a position of the distal end of the instrument.
